(19) United States
(12) Patent Application Publication
(10) Pub. No.: US 2011/0287527 A1
(43) Pub. Date: Nov. 24, 2011

(54) Gas treatment appliance and gas treatment process

(75) Inventor: Peter Lutz, Munich (DE)

(73) Assignee: BEKON Energy Technologies GmbH & Co. KG

(21) Appl. No.: 13/135,007

(22) Filed: Jun. 23, 2011

Related U.S. Application Data
(63) Continuation of application No. PCT/EP2009/067865, filed on Dec. 23, 2009.

(30) Foreign Application Priority Data
Dec. 23, 2008 (DE) DE 102008062811.5

(51) Int. Cl. A61L 9/01; C12M 1/04

(52) U.S. Cl. 435/266; 435/289.1

(57) ABSTRACT

A gas treatment appliance and a gas treatment process are provided. The proportion of tar is decreased in a product gas formed on the gasification of carbonaceous feedstocks. The product gas or synthesis gas formed on the gasification of carbonaceous feedstocks is generally highly polluted with tar. This tar-polluted gas is exposed to a solid support substrate which is a support for bacteria which degrade tar. Suitable bacteria are, in particular, bacteria that occur in biogas-generating processes. The bacteria contained in the solid support substrate convert at least some of the tars and polycyclic aromatic hydrocarbons (PAHs) contained in the product gas into shorter-chain hydrocarbon compounds and methane which are not an industrial problem, and so their proportion is reduced in the product gas leaving the support substrate. In this manner the tar pollution in a synthesis gas or product gas can be reduced in a simple and inexpensive manner.

### CROSS REFERENCE TO RELATED APPLICATION

This application is filed under 35 U.S.C. §111(a) and is based on and hereby claims priority under 35 U.S.C. §120 and §365(c) from International Application No. PCT/EP2009/067865, filed on Dec. 23, 2009, and published as WO 2010/072818 A2 on Jul. 1, 2010, which in turn claims priority from German Application No. 102008062811.5, filed on Dec. 23, 2008, in Germany. This application is a continuation of International Application No. PCT/EP2009/067865, which is a continuation of German Application No. 102008062811.5. International Application No. PCT/EP2009/067865 is pending as of the filing date of this application, and the United States is an elected state in International Application No. PCT/EP2009/067865. This application claims the benefit under 35 U.S.C. §119 from German Application No. 102008062811.5. The disclosure of each of the foregoing documents is incorporated herein by reference.

### TECHNICAL FIELD

The present invention relates to a gas treatment appliance and a gas treatment process for the reduction of the proportion of tar in a product gas that is created upon gasification of the carbonaceous feedstocks.

### BACKGROUND

The thermal gasification of carbonaceous feedstocks and in particular wood gasification is a procedurally employed chemical reaction, which allows, by means of pyrolysis, which is to say the thermal splitting of chemical compounds, or partial combustion of the carbonaceous feedstocks under conditions of oxygen deficiency to achieve a burnable product gas, in the case of wood, the so-called wood gas. Wood gas, among other things, was and is used to operate automobiles or combustion engines, which are coupled with a generator, which in turn produces power. In the case of gasification, the wood that functions as fuel is introduced in an under-stoichiometric amount to an oxidization agent (generally air, less commonly water vapor). In so doing part of the wood is burnt. The heat that is thereby created is then used for the thermal degradation of the remaining wood quantity. The wood gas that is produced by gasification essentially delivers a gas mixture of hydrogen (H₂), Carbon monoxide (CO), Carbon dioxide (CO₂), water vapor, Methane (CH₄) and atmospheric nitrogen (N₂). It is even possible to obtain a burnable gas through thermal gasification starting off with coal.

A disadvantage does result, insofar that as a result of an incomplete gasification, the product gas/wood gas contains a greater or lesser quantity of unwanted tar compounds (higher boiling hydrocarbons) and polycyclic aromatic hydrocarbons (PAH).

In the case of use with an Otto gas engine, as an example, the functionality of the gas delivery system (mixer, valves, and sensor) was impacted by these compounds by means of condensation and especially by way of resublimation. Furthermore, the burning of the wood gas polluted with'tar easily led to the exhaust emissions to exceed the limits set forth by the TA Luft (“Technical Instructions on Air Quality Control”) for motor assemblies.

A device for the increase of the gas yield of a biogas plant according to the principle of liquid fermentation is known from DE 20 2004 014 510 U1. The product gas polluted with tar is piped through a wood gasifier in the liquid biomass which is in the form of liquid manure. Insofar as there are also bacteria that degrade the tar contained in the liquid manure, the tar that is found in the wood gas is partially degraded. However the degradation is not terrible effective, insofar as only a limited proportion of the bacteria contained in the liquid manure is able to degrade tar, whereas the largest proportion of the bacteria is “specialized” in the degradation of biomass. Insofar as it is systemic (given that one is dealing with a biogas plant) that there is sufficient biomass available, it is also not possible for the bacteria that degrade the tar to automatically enrich themselves.

There is a selection procedure for bacteria strains that is known from DE 195 15 578 A1, in which targeted undesired bacteria are killed off in a mixture of bacteria.

It is therefore a purpose of the present invention to provide a gas treatment appliance and a gas treatment process, in which the proportion of tar in a product gas that is created upon gasification of carbonaceous feedstocks is effectively reduced.

### SUMMARY

Embodiments of the present invention provide a gas treatment appliance and a gas treatment process, in which the proportion of tar in a product gas that is created upon gasification of carbonaceous feedstocks is effectively reduced.

In one embodiment, a gas treatment appliance for the reduction of the proportion of tar in a product gas that is formed upon the gasification of carbonaceous is provided. The gas treatment appliance comprises a fixed bed reactor that works by the principle of dry fermentation, in which a solid support substrate is located and which contains bacteria which degrade the tar; a feeder line for the tar polluted product gas (HG_{TT}); an outlet line for the processed product gas (HG_{T}); a percolate circuit; a gas-cooling appliance for the cooling of the tar polluted product gas (HG_{TT}) placed prior to its introduction into the fixed bed reactor; and a condensate introduction device, by wherein a condensate occurring during cooling is introduced to the solid support substrate of the fixed bed reactor through the percolate circuit.

In another embodiment, a gas treatment process for the reduction of the proportion of tar in a product gas that is created upon gasification of carbonaceous feedstocks is provided. The gas treatment process is performed by means of a gas treatment appliance. A tar-polluted product gas (HG_{TT}) is first created upon gasification of carbonaceous feedstocks. The tar-polluted product gas (HG_{TT}) is then cooled by a gas-cooling appliance, while a condensate introduction device is used to generate a condensate occurring during the cooling. The cooled tar-polluted product gas (HG_{TT}) is then introduced into a fixed bed reactor that works by the principle of dry fermentation. The fixed bed reactor contains a solid support substrate. The condensate occurring is introduced to the solid support substrate through a percolate circuit. Finally, the tar polluted product gas (HG_{TT}) is perfused through the solid support substrate made up of not readily biodegradable material that degrades tar.

Other embodiments and advantages are described in the detailed description below. This summary does not purport to define the invention. The invention is defined by the claims.

### BRIEF DESCRIPTION OF THE DRAWINGS

The accompanying drawings, where like numerals indicate like components, illustrate embodiments of the invention.

FIG. 1 shows a gas treatment appliance in a schematic fashion according to one embodiment of this invention.

### DETAILED DESCRIPTION

Reference will now be made in detail to some embodiments of the invention, examples of which are illustrated in the accompanying drawings.

Embodiments of the present invention provide a gas treatment appliance and a gas treatment process, in which the proportion of tar in a product gas that is created upon gasification of carbonaceous feedstocks is effectively reduced. This purpose is solved through the features of claim 1 or claim 10.

The product gas or synthesis gas, which is created by gasification of carbonaceous feedstocks, is generally heavily polluted with tar. This product gas that is polluted with tar is exposed to a solid support substrate in a fixed bed reactor, whose support material is made up of bacteria, which degrade the tar. Such bacteria are especially contained in bacterial mixture populations, such as those which occur in processes which generate biogas. The bacteria that are contained in the solid support substrate do convert at least a portion of the tar and the polycyclic aromatic hydrocarbons (PAH) that are contained in the product gas into technically uncomplicated, shorter chain hydrocarbon compounds and methane, so that their proportion in the outgoing product gas leaving the solid support substrate is reduced. Insofar as the bacteria mixture population is established on a solid support substrate, there is no readily biodegradable material available for the bacteria. The nourishment that is available is the tar in the product gas that is to be cleaned. As a result, the bacteria which cannot process any tar die off, and the solid support substrate enriches itself with bacteria that degrade tar in such a manner that the filter function of the gas treatment appliance is improved. Bacteria that degrade tar generally thrive in an aerobic environment, bacteria that can degrade tar in an aerobic environment are however imaginable. The load of tar can thereby be reduced in a simple and inexpensive manner to a synthesis or product gas.

Using the advantageous embodiments according to claims 2 and 3, many bacteria in or on the solid support substrate potentially come into contact with the product gas polluted with tar.

A fill represents a simple and inexpensive embodiment of a solid support substrate—claim 3.

Using the advantageous embodiment according to claim 4, the selection pressure is further strengthened in the direction of enrichment of the bacteria that degrade tar, insofar as the Supply of nourishment other than the tar is reduced.

The object of claim 5 presents particularly suitable material that is not readily biodegradable for the solid support substrate.

Using the preferred embodiment of the invention according to claim 6, the fill is prepared in a simple way with bacteria that degrade the tar. Spent biomass that stems for a biogas plant contains mainly bacteria that can degrade biomass, however it also contains bacteria that can degrade tar. Insofar as there is no fresh biomass in the gas treatment appliance according to the invention, but rather above all else tar is “offered”, the bacteria that degrade the tar are multiplied, whereas the portion of bacteria that do not degrade the tar diminishes. The suitable bacteria are therefore automatically selected from the bacteria cocktail and are enriched. The organic portion of household garbage that is “spent” according to the principle of dry fermentation by a biogas plant is suitable as the solid support substrate loaded with bacteria that degrade tar. This spent biomass is already so terribly polluted with pollutants that a disposal by means of composting already does not readily come into the question.

Using the embodiment according to claim 7, or respectively claim 13, prevents that the bacteria that degrade the tar are either damaged or destroyed in the short routings of the hot product gas (up to 800° C.). The tar that occurs during the cooling to a liquid form is introduced by means of a condensate introduction device on the solid support substrate with the bacteria that degrade the tar (claim 8).

Temperature, as well as also the level of humidity of the solid support substrate (of the environment) are deciding parameters, which have an influence on the conversion process that takes place in the gas treatment containers and thereby on the degree of efficiency of the treatment. By way of the wetting done according to claim 9 or respectively claim 11, it is prevented that there is a partial drying of the solid support substrate and the thereby associated inactivation of the bacteria that degrade the tar. This wetting takes place in an advantageous manner by means of a percolate circuit, as is known from the principle of dry fermentation in biogas plants. It can also be advantageous in the case of certain solid support substrates and bacteria that degrade the tar that there be a complete flooding of the solid support substrate with the bacteria that degrade the tar.

It can also be advantageous in certain solid support substrates and bacteria that degrade the tar that there be a complete flooding of the solid support substrate with percolate (claim 12).

Using the advantageous embodiment of the invention according to claim 14 one is spared the use of a condensate introduction device. A mixing of both liquids is achieved in a procedurally simple manner by means of introduction of the condensate into the percolate, prior to the same having been introduced into the gas treatment container after having been oozed in and oozed out of the gas treatment container.

Further details, features and advantages of this invention will become evident upon reading the following detailed description of a preferred embodiment according to the attached drawing.

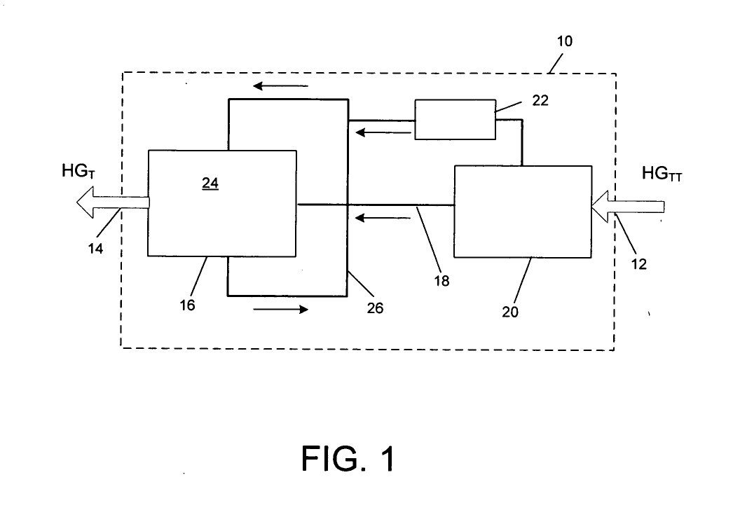

FIG. 1 shows a gas treatment appliance in a schematic fashion according to one embodiment of this invention.

FIG. 1 shows a gas treatment appliance 10 according to the invention for the reduction of the proportion of tar in a product gas, whereby the product gas is wood gas in accordance with the embodiment, that is created during the allothermal water vapor gasification of wood. The gas treatment appliance 10 is made up of a wood gas inlet line 12 for the delivery of a tar polluted wood gas HG_{TT} and a wood gas outlet line 14 for the discharge of a processed wood gas HG_{T}, which is to say having a lesser load of tar. The gas treatment appliance furthermore includes a fixed bed reactor 16 and a gas cooling device 20 that is linked to the same by way of a connector line 18. The wood gas HG_{TT} that is introduced into the gas treatment appliance 10 by way of the wood gas input line 12 is cooled in the gas cooling device 20, introduced by means of the connector line 18 to the fixed bed reactor 16, where the tar is at least partially removed, in such a manner that the proportion of'tar therein is reduced, and is discharged from the gas treatment appliance 10 as wood gas HG_{T} which is less polluted with tar. Furthermore, a predetermined percentage of the condensate that occurs during cooling in the gas cooling device 20 is introduced to a percolate circuit 26 by way of a condensate introduction device 22. The percolate circuit 26 is then fed percolate from a biogas plant working according to the principle of dry fermentation. This percolate is an aqueous solution which is enriched with a mixed bacteria population which also contains bacteria that degrade tar. This percolate is used to continually or intermittently wet or flood the solid support substrate. The percolate is extracted from the fixed bed reactor 16 and then fed once again to the same, which is to say re-circulated, as is schematically demonstrated in FIG. 1 by a pipeline 26.

Insofar as the solid support substrate 24 is made up of materials that are poorly or alternatively difficult to exploit in or alternatively on the solid support substrate 24, all such bacteria that are not able to degrade tar die off, whereas the bacteria that can degrade tar are enriched. Cellulose (wood chips or straw), porous ceramic, plastics and in particular spent biomass from a biogas plant working on the dry fermentation principle are suitable material for the solid support substrate 24.

### LIST OF REFERENCE SYMBOLS

10 Gas treatment appliance
12 Feeder line
14 Outlet line
16 Fixed bed reactor
18 Connector line
20 Gas cooling device
22 Condensate introduction device
24 Bacteria polluted solid support substrate
26 Percolate circuit
HG_{T} Tar polluted wood gas
HG_{TT} Processed wood gas

Although the present invention has been described in connection with certain specific embodiments for instructional purposes, the present invention is not limited thereto. Accordingly, various modifications, adaptations, and combinations of various features of the described embodiments can be practiced without departing from the scope of the invention as set forth in the claims.

1-15. (canceled)

16. A gas treatment appliance (10) for reduction of the proportion of tar in a product gas, which is formed upon the gasification of carbonaceous feedstocks, the gas treatment appliance (10) comprises:
a fixed bed reactor (16) that works by the principle of dry fermentation, in which a solid support substrate (24) is located and which contains bacteria which degrade the tar;
a feeder line (12) for the tar polluted product gas (HG_{TT});
an outlet line (14) for the processed product gas (HG_{T});
a percolate circuit (26);
a gas-cooling appliance (20) for cooling of the tar polluted product gas (HG_{TT}) placed prior to its introduction into the fixed bed reactor; and
a condensate introduction device (22), wherein a condensate occurring during cooling is introduced to the solid support substrate (24) of the fixed bed reactor (16) through the percolate circuit (26).

17. The gas treatment appliance (10) of claim 16, wherein the manner in which the feeder line (12) and the outlet line (14) are arranged so that the product gas is perfused through the solid support substrate (24).

18. The gas treatment appliance (10) of claim 16, wherein the solid support substrate (24) being a fill.

19. The gas treatment appliance (10) of claim 16, wherein the solid support substrate (24) is made up of not readily biodegradable materials.

20. The gas treatment appliance (10) of claim 19, wherein the solid support substrate (24) is made up of ceramic, woodchips, straw, and/or plastic bodies.

21. The application of the gas treatment appliance (10) of claim 16, wherein the solid support substrate (24) being a fill, and wherein the fill being made up of spent biomass with tar degrading bacteria coming from a biogas plant, which works according to the principle of dry fermentation.

22. A gas treatment process for reduction of the proportion of tar in a tar polluted product gas (HG_{TT}) by means of a gas treatment appliance, the gas treatment process comprises:
receiving the tar polluted product gas (HG_{TT}) that is formed upon gasification of carbonaceous feedstocks; and
perfusing the tar polluted product gas (HG_{TT}) through a solid support substrate (24) made up of not readily biodegradable material that degrades tar.

23. The gas treatment process of claim 22, wherein the gas treatment appliance being set out in the style of a biogas plant, in which the solid support substrate (24) is wetted according to the principle of dry fermentation by means of a percolate circuit (26).

24. The gas treatment process (10) of claim 22, wherein the solid support substrate (24) is flooded with percolate.

25. The gas treatment process (10) of claim 22, wherein the product gas being cooled prior to the introduction to the solid support substrate (24).

26. The gas treatment process (10) of claim 25, wherein a condensate that arises upon cooling is introduced to the solid support substrate (24) through a percolate circuit (26).

27. The gas treatment process of claim 22, wherein the product gas being wood gas.

28. A method, comprising:
receiving a tar polluted product gas (HG_{TT}) that is formed upon gasification of carbonaceous feedstocks;
cooling the tar polluted product gas (HG_{TT}) by a gas-cooling appliance, wherein a condensate introduction device is used and thereby generating a condensate occurring during the cooling;
introducing the cooled tar polluted gas (HG_{TT}) into a fixed bed reactor that works by the principle of dry fermentation, wherein a solid support substrate is located within the fixed bed reactor; and
perfusing the tar polluted product gas (HG_{TT}) through the solid support substrate made up of not readily biodegradable material that degrades tar.

29. The method of claim 28, wherein the solid support substrate is made up of ceramic, woodchips, straw, and/or plastic bodies.

30. The method of claim 28, wherein the solid support substrate being a fill, and wherein the fill being made up of spent biomass with tar degrading bacteria coming from a biogas plant that works according to the principle of dry fermentation.

31. The method of claim 28, wherein the solid support substrate is flooded with percolate.

32. The method of claim 28, wherein the condensate occurring that arises upon the cooling is introduced to the solid support substrate through a percolate circuit.

33. The method of claim 28, wherein the product gas being wood gas.
